(19) United States
(12) Patent Application Publication
(10) Pub. No.: US 2008/0226759 A1
(43) Pub. Date: Sep. 18, 2008

(54) SYSTEM FOR TREATING ADDICTIONS

(76) Inventor: Yakov Marshak, Irvine, CA (US)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 11/686,886

(22) Filed: Mar. 15, 2007

(51) Int. Cl. A61K 36/30; A61K 31/122; A61K 31/131; A61K 31/145; A61M 16/00; A61P 43/00; A61K 31/195; A61K 31/435; A61K 36/00

(52) U.S. Cl. 424/776; 128/204.18; 424/725; 514/295; 514/354; 514/567; 514/665; 514/680; 514/789

(57) ABSTRACT

A system for treating addictions includes genetic testing to determine the presence of allelic variants of genes shown to be associated with impulsive/addictive behaviors and a tailored rehabilitation regimen of specific physical exercise, special diet and particular dietary supplements to optimize the way mood elevation is achieved in the patient. The system further includes individual and group counseling and initial oxygen therapy. The genetic testing assesses the presence of gene polymorphism in at least one of D2 dopamine receptor gene (DRD2), dopamine transporter gene (DAT1), dopamine beta-hydroxylase gene (DBH) and serotonin transporter (SERT) gene. The dietary supplements are tailored to stimulate production of neuromediators or neurotransmitters, depending upon the polymorphism determined. The exercises include static and dynamic routines also tailored to the results of the genetic testing. After completion of the inpatient portion of the system, further therapies include daily exercise and dietary supplement programs tailored for each polymorphism discovered.

### FIELD OF INVENTION

The invention is a methodology for treating drug, alcohol and other addictions. More particularly, the methodology involves rapid elevation and stabilization of the sense of well being in patients that ultimately leads to a reduction in drug craving and drug seeking behavior.

### BACKGROUND OF THE INVENTION

Alcohol and drug addictions can have a pervasive and devastating effect on the lives of those suffering these conditions. Due to the combined physical and psychological aspects of these addictions, they can be extremely difficult to overcome and incidents of individuals returning to the addictive behaviors tend to be high. In recent times, the study of genetics has pointed up links between certain gene characteristics and tendencies to addiction. Various methods have been developed to identify these genes and to tailor treatments for individuals having these genetic characteristics.

U.S. Pat. No. 5,500,343, issued to Blum, et al., is directed to a method for detecting compulsive disorder susceptibility of a human. The method comprises initially obtaining a DNA sample of a human and then determining the presence or absence of a particular D2 receptor gene allele. Detection of the allele in the sample is indicative of susceptibility to compulsive disorder (mainly alcohol and cocaine). The human dopamine D2 receptor gene allele may be an A1 allele, a B1 allele, or a DRD2 allele. In Table 1 of the reference, the allelic association with substance abuse is shown. The reference alludes to studies that may identify polymorphisms that exist in the gene encoding the 5HT-2 (serotonin) receptor.

U.S. Pat. No. 6,955,873, issued to Blum, is directed to a kit and an intravenously administrable preparation, both, with a signal transmitter precursor, an enhancer of precursor uptake, and an inhibitor of neural transmitter reuptake or signal transmitter catabolism. Either the kit composition or the IV formulation may be used as guided by a subject's allelic analysis. The important alleles, as indicated by the reference, include the DRD2 gene, the DAT1 gene and the DBH gene. The reference states that those alleles, among others mentioned, indicate at least one RDS behavior (reward deficiency syndrome). The reference further discloses a composition for the treatment of RDS behaviors in a subject. The reference also discloses a treatment of the RDS behavior using an RDS nutraceutical and selection of herbal or other remedies to ameliorate the specific RDS behavior. The reference also discloses forms in Tables 1-3 used for diagnosis in designing a rehabilitation program for a patient.

U.S. Pat. No. 6,001,848, issued to Noble, illustrates compounds and therapeutic kits useful in the treatment of alcoholics having the A1 allele of the dopamine receptor D2G. Also disclosed are methods of treating alcoholics having the A1/A1 or A1/A2 DRD2 genotype comprising administration of dopamine agonists. The disclosed method involves identifying an alcoholic subject having a DRD2 A1 allele; and administering to the subject an amount of a DRD2-specific dopamine agonists composition sufficient to alleviate the alcohol addiction. The preferred dopamine agonists include ergolines or aporphines.

U.S. Pat. No. 6,908,631, issued to Sellers et al., disclose a method of regulating the activity of human cytochrome P450 isozyme CYP2A6 to control nicotine metabolism or decrease the production of carcinogens from procarcinogens, such as those present in tobacco smoke, in an individual by selectively inhibiting CYP2A6. The reference discloses that the presence in an individual of a mutant allele of human cytochrome P450 enzyme CYP2A6 is predictive of an individual who: (1) has a decreased risk of becoming a smoker, (2) will smoke less if he/she becomes dependent, and/or (3) may be at relatively lower risk for cancer due to both decreased smoke exposure and decreased CYP2A6-mediated activation of tobacco smoke and other procarcinogenic substrates. The reference discloses that once an individual is identified, he/she may be treated prophylactively with effective quantities of CYP2A6 inhibitors.

U.S. Pat. No. 6,165,716, issued to Battersby et al., is directed to screening for disorders of serotonergic dysfunction. The reference discloses three novel alleles of the serotonin transporter gene that are effective markers for screening and diagnosis of migraine and psychiatric disorders. The reference also teaches methods for in vitro screening of individuals using DNA taken from blood samples. Although the reference discloses specific sequence IDs, such does disclose the correlation between the alleles of the serotonin transporter gene and various psychiatric disorders (compulsion being but one example).

It is an objective of the present invention to provide a method for identifying genetic characteristics linked to addictive behavior. It is a further objective to provide a series of mood-elevating counseling treatments for individuals exhibiting these genotypes that are tailored to their particular genetic makeup. It is yet a further objective to provide a dietary plan matched to the genetic makeup of the individual undergoing treatment. It is still a further objective to determine herbal supplements and physical exercises that will complement these genotype specific treatments. Finally, it is an objective of the present invention to provide oxygen therapy and specific hygienic procedures during the first days of adaptation/transition period.

While some of the objectives of the present invention are disclosed in the prior art, none of the inventions found include all of the requirements identified.

### SUMMARY OF THE INVENTION

The present invention addresses all of the deficiencies of prior art systems for treating addictions and satisfies all of the objectives described above.

(1) A system for treating addictions providing the desired features may be constructed from the following components. Genetic testing of a patient to determine the presence of allelic variants of genes shown to be associated with impulsive/addictive behaviors is provided. A rehabilitation regimen of specific physical exercise, special diet and particular dietary supplements is provided, to optimize the way mood elevation is achieved in the patient.

(2) In a variant of the invention, the system further includes a psychological rehabilitation program.

(3) In another variant, the psychological rehabilitation program includes individual and group counseling.

(4) In still another variant, the genetic testing assesses the presence of gene polymorphism in at least one of D2 dopamine receptor gene (DRD2), dopamine transporter gene (DAT1), dopamine beta-hydroxylase gene (DBH) and serotonin transporter (SERT) gene.

(5) In yet another variant, the specific physical exercises are adapted from at least one of Kudalini yoga, Hatha yoga, T'ai Chi Ch'üan and holotropic breathing.

(6) In still another variant, the specific physical exercises are performed in morning, afternoon and evening sessions before eating.

(7) In a further variant, the specific physical exercises performed in morning and afternoon sessions comprise dynamic physical exercises that include rhythmically repeated movements combined with hyperventilation, contraction of abdominal muscles and inferior and superior small pelvis diaphragm muscles, alternated with muscle relaxation and deep diaphragm-type breathing designed to elevate the patient's mood by producing a feeling of mental and physical enhancement in an invigorating, energizing manner.

(8) In still a further variant, the specific physical exercises performed in evening sessions comprise static exercises that are based on posture maintenance, stretching of muscles and tendons, combined with relaxation and deep diaphragm-type breathing.

(9) In another variant of the invention, the static exercises comprise stretching the patient's body outwardly to a point of feeling light pain followed by muscle relaxation with deep breathing until the pain eases. The exercise cycle repeated at least once for each of the stretching exercises.

(10) In still another variant, the special diet comprises carbohydrates that have a low glycemic index combined with fats and proteins and carbohydrates that have a medium glycemic index combined with proteins and vegetable fibers.

(11) In yet another variant, prior to analysis of the genetic testing, the particular dietary supplements comprise at least one of D, L phenylalanine, L-tyrosine, dimethyl-amino-ethanol (DMAE), 5-hydroxytryptophan (5HTP), hypericin (from St John's wort extract), vitamin B6, L-theanine, taurine, huperzine A, borage oil, and a vitamin and mineral complex.

(12) In a further variant, after analysis of the genetic testing, for clients who have been detected with at least one of the following alleles: the A1 allele of D2 dopamine receptor gene (Taq1 polymorphism), the nine-repeat allele of DAT1 dopamine transporter gene (variable number of tandem repeats (VNTR) polymorphism of 3′ untranslated region), the short allele of SERT serotonin transporter gene (44-base-pair insertion/deletion polymorphism of the promoter region), the particular dietary supplements, which increase the production of neuromediators, comprise at least one of 5-hydroxytryptophan (5HTP), hypericin (from St John's wort extract), vitamin B6, L-dopa (from Mucuna Pruriens), D, L phenylalanine, L-theanine, taurine, huperzine A, borage oil, and a vitamin and mineral complex.

(13) In still a further variant, after analysis of the genetic testing, for clients who have been detected with A2 allele of Taq1 polymorphism in the DBH dopamine beta-hydroxylase gene, the particular dietary supplements, which increase the production of the neurotransmitters, comprise at least one of D, L phenylalanine, L-tyrosine, dimethyl-amino-ethanol (DMAE), L-DOPA (from Mucuna pruriens), L-theanine, taurine, borage oil, and a vitamin and mineral complex.

(14) In yet a further variant, the system for treating addictions further includes at least one oxygen therapy treatment.

(15) In another variant of the invention, the at least one oxygen therapy treatment comprises air enriched with oxygen so that the air includes 28%-40% oxygen.

(16) In yet another variant, the at least oxygen therapy treatment comprises air enriched with oxygen provided to the patient at a rate of 2-6 liters per minute.

(17) In still another variant, the patient performs the dynamic physical exercises in morning sessions during an initial period after completion of the system's implementation.

(18) In a further variant, the dynamic exercises are preceded by consumption of the particular dietary supplements.

(19) In yet a further variant, the patient performs the static physical exercises in evening sessions during an initial period after completion of the system's implementation.

(20) In still a further variant, the static exercises are preceded by consumption of the particular dietary supplements.

(21) In another variant, patients with polymorphism in a DBH dopamine beta-hydroxylase gene perform only the dynamic exercises after an initial period following completion of the system's implementation.

(22) In still another variant, patients with polymorphism in any of DRD2 dopamine D2 receptor, DAT dopamine transporter, or SERT serotonin transporter genes perform only the static exercises after an initial period following completion of the system's implementation.

(23) In yet another variant, the dynamic exercises are performed in a morning session and a short version of static exercises are performed in an evening session.

(24) In a still another variant of the invention, the static exercises are performed in an evening session and a short version of dynamic exercises are performed in a morning session.

(25) In a further variant, said particular dietary supplements, which increase the production of neuromediators, are taken after an initial period following completion of said system's implementation.

(26) In a final variant, said particular dietary supplements, which increase the production of the neurotransmitters, are taken after an initial period following completion of said system's implementation.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a schematic view of the features and relative timing of the Inpatient Program and the Post Completion Therapy.

### DETAILED DESCRIPTION OF THE PREFERRED EMBODIMENT

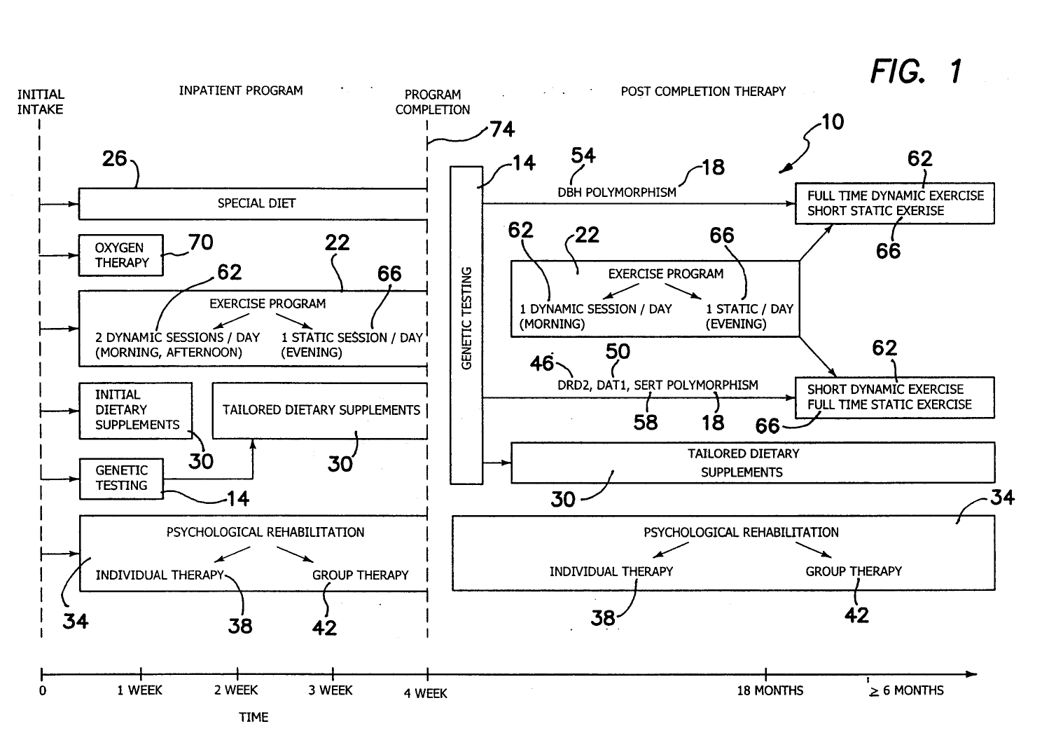

(1) FIG. 1 illustrates a system for treating addictions 10 providing the desired features that may be constructed from the following components. Genetic testing 14 of a patient to determine the presence of allelic variants of genes shown to be associated with impulsive/addictive behaviors 18 is provided. A rehabilitation regimen of specific physical exercise 22, special diet 26 and particular dietary supplements 30 is provided, to optimize the way mood elevation is achieved in the patient.

(2) In a variant of the invention, the system further includes a psychological rehabilitation program 34.

(3) In another variant, the psychological rehabilitation program 30 includes individual 38 and group 42 counseling.

(4) In still another variant, the genetic testing 14 assesses the presence of gene polymorphism 18 in at least one of D2 dopamine receptor gene (DRD2) 46, dopamine transporter gene (DAT1) 50, dopamine beta-hydroxylase gene (DBH) 54 and serotonin transporter (SERT) gene 58.

(5) In yet another variant, the specific physical exercises 22 are adapted from at least one of Kudalini yoga, Hatha yoga, T'ai Chi Ch'üan and holotropic breathing.

(6) In still another variant, the specific physical exercises 22 are performed in morning, afternoon and evening sessions before eating.

(7) In a further variant, the specific physical exercises 22 performed in morning and afternoon sessions comprise dynamic physical exercises 62 that include rhythmically repeated movements combined with hyperventilation, contraction of abdominal muscles and inferior and superior small pelvis diaphragm muscles, alternated with muscle relaxation and deep diaphragm-type breathing designed to elevate the patient's mood by producing a feeling of mental and physical enhancement in an invigorating, energizing manner.

(8) In still a further variant, the specific physical exercises 22 performed in evening sessions comprise static exercises 66 that are based on posture maintenance, stretching of muscles and tendons, combined with relaxation and deep diaphragm-type breathing.

(9) In another variant of the invention, the static exercises 66 comprise stretching the patient's body outwardly to a point of feeling light pain followed by muscle relaxation with deep breathing until the pain eases. The exercise cycle repeated at least once for each of the stretching exercises.

(10) In still another variant, the special diet 26 comprises carbohydrates that have a low glycemic index combined with fats and proteins and carbohydrates that have a medium glycemic index combined with proteins and vegetable fibers.

(11) In yet another variant, prior to analysis of the genetic testing 14, the particular dietary supplements 30 comprise at least one of D, L phenylalanine, L-tyrosine, dimethyl-amino-ethanol (DMAE), 5-hydroxytryptophan (5HTP), hypericin (from St John's wort extract), vitamin B6, L-theanine, taurine, huperzine A, borage oil, and a vitamin and mineral complex.

(12) In a further variant, after analysis of the genetic testing 14, for clients who have been detected with at least one of the following alleles: the A1 allele of D2 dopamine receptor gene 46 (Taq1 polymorphism), the nine-repeat allele of DAT1 dopamine transporter gene 50 (variable number of tandem repeats (VNTR) polymorphism of 3′ untranslated region), the short allele of SERT serotonin transporter gene 58 (44-base-pair insertion/deletion polymorphism of the promoter region), the particular dietary supplements 30, which increase the production of neuromediators, comprise at least one of 5-hydroxytryptophan (5HTP), hypericin (from St John's wort extract), vitamin B6, L-dopa (from Mucuna Pruriens), D, L phenylalanine, L-theanine, taurine, huperzine A, borage oil, and a vitamin and mineral complex.

(13) In still a further variant, after analysis of the genetic testing 14, for clients who have been detected with A2 allele of Taq1 polymorphism in the DBH dopamine beta-hydroxylase gene 54, the particular dietary supplements, which increase the production of the neurotransmitters, comprise at least one of D, L phenylalanine, L-tyrosine, dimethyl-amino-ethanol (DMAE), L-DOPA (from Mucuna pruriens), L-theanine, taurine, borage oil, and a vitamin and mineral complex.

(14) In yet a further variant, the system for treating addictions further includes at least one oxygen therapy treatment 70.

(15) In another variant of the invention, the at least one oxygen therapy treatment 70 comprises air enriched with oxygen so that the air includes 28%-40% oxygen.

(16) In yet another variant, the at least oxygen therapy treatment 70 comprises air enriched with oxygen provided to the patient at a rate of 2-6 liters per minute.

(17) In still another variant, the patient performs the dynamic physical exercises 62 in morning sessions during an initial period after completion of the system's implementation 74.

(18) In a further variant, the dynamic exercises 62 are preceded by consumption of the particular dietary supplements 30.

(19) In yet a further variant, the patient performs the static physical exercises 66 in evening sessions during an initial period after completion of the system's implementation 74.

(20) In still a further variant, the static exercises 66 are preceded by consumption of the particular dietary supplements 30.

(21) In another variant, patients with polymorphism 18 in a DBH dopamine beta-hydroxylase gene 54 perform only the dynamic exercises 62 after an initial period following completion of the system's implementation 74.

(22) In still another variant, patients with polymorphism 18 in any of DRD2 dopamine D2 receptor 46, DAT dopamine transporter 50, or SERT serotonin transporter 58 genes perform only the static exercises 66 after an initial period following completion of the system's implementation 74.

(23) In yet another variant, the dynamic exercises 62 are performed in a morning session and a short version of static exercises 66 are performed in an evening session.

(24) In a still another variant of the invention, the static exercises 66 are performed in an evening session and a short version of dynamic exercises 62 are performed in a morning session.

(25) In a further variant, said particular dietary supplements 30, which increase the production of neuromediators, are taken after an initial period following completion of said system's implementation 74.

(26) In a final variant, said particular dietary supplements 30, which increase the production of the neurotransmitters, are taken after an initial period following completion of said system's implementation 74.

The system for treating addictions 10 has been described with reference to particular embodiments. Other modifications and enhancements can be made without departing from the spirit and scope of the claims that follow.

1. A system for treating addictions, comprising:
genetic testing of a patient to determine the presence of allelic variants of genes shown to be associated with impulsive/addictive behaviors;
a rehabilitation regimen of specific physical exercise, special diet and particular dietary supplements, to optimize the way mood elevation is achieved in said patient.

2. The system for treating addictions, as described in claim 1, further comprising a psychological rehabilitation program.

3. The system for treating addictions, as described in claim 2, wherein said psychological rehabilitation program includes individual and group counseling.

4. The system for treating addictions, as described in claim 1, wherein said genetic testing assesses the presence of gene polymorphism in at least one of D2 dopamine receptor gene (DRD2), dopamine transporter gene (DAT1), dopamine beta-hydroxylase gene (DBH) and serotonin transporter (SERT) gene.

5. The system for treating addictions, as described in claim 1, wherein said specific physical exercises are adapted from at least one of Kudalini yoga, Hatha yoga, T'ai Chi Ch'üan and holotropic breathing.

6. The system for treating addictions, as described in claim 1, wherein said specific physical exercises are performed in morning, afternoon and evening sessions before eating.

7. The system for treating addictions, as described in claim 1, wherein said specific physical exercises performed in morning and afternoon sessions comprise dynamic physical exercises that include rhythmically repeated movements combined with hyperventilation, contraction of abdominal muscles and inferior and superior small pelvis diaphragm muscles, alternated with muscle relaxation and deep diaphragm-type breathing designed to elevate said patient's mood by producing a feeling of mental and physical enhancement in an invigorating, energizing manner.

8. The system for treating addictions, as described in claim 1, wherein said specific physical exercises performed in evening sessions comprise static exercises that are based on posture maintenance, stretching of muscles and tendons, combined with relaxation and deep diaphragm-type breathing.

9. The system for treating addictions, as described in claim 8, wherein said static exercises comprise stretching said patient's body outwardly to a point of feeling light pain followed by muscle relaxation with deep breathing until said pain eases, said exercise cycle repeated at least once for each of said stretching exercises.

10. The system for treating addictions, as described in claim 1, wherein said special diet comprises:
carbohydrates having a low glycemic index combined with fats and proteins; and
carbohydrates having a medium glycemic index combined with proteins and vegetable fibers.

11. The system for treating addictions, as described in claim 1, wherein, prior to analysis of said genetic testing, said particular dietary supplements comprise at least one of:
D, L phenylalanine;
L-tyrosine;
dimethyl-amino-ethanol (DMAE);
5-hydroxytryptophan (5HTP);
hypericin (from St John's wort extract);
vitamin B6;
L-theanine;
taurine;
huperzine A;
borage oil; and
vitamin and mineral complex.

12. The system for treating addictions, as described in claim 1, wherein, after analysis of said genetic testing, clients who have been detected with at least one of the following alleles: the A1 allele of D2 dopamine receptor gene (Taq1 polymorphism), the nine-repeat allele of DAT1 dopamine transporter gene (variable number of tandem repeats (VNTR) polymorphism of 3′ untranslated region), the short allele of SERT serotonin transporter gene (44-base-pair insertion/deletion polymorphism of the promoter region), said particular dietary supplements, which increase the production of neuromediators, comprise at least one of:
5-hydroxytryptophan (5HTP);
hypericin (from St John's wort extract);
vitamin B6;
L-dopa (from Mucuna Pruriens);
D, L phenylalanine;
L-theanine;
taurine;
huperzine A;
borage oil; and
vitamin and mineral complex.

13. The system for treating addictions, as described in claim 1, wherein, after analysis of said genetic testing, clients who have been detected with A2 allele of Taq1 polymorphism in said DBH dopamine beta-hydroxylase gene, said particular dietary supplements, which increase the production of the neurotransmitters, comprise at least one of:
D, L phenylalanine;
L-tyrosine;
dimethyl-amino-ethanol (DMAE);
L-DOPA (from Mucuna pruriens);
L-theanine;
taurine;
borage oil; and
vitamin and mineral complex.

14. The system for treating addictions, as described in claim 1, further comprising at least one oxygen therapy treatment.

15. The system for treating addictions, as described in claim 14, wherein said at least one oxygen therapy treatment comprises air enriched with oxygen so that said air includes 28%-40% oxygen.

16. The system for treating addictions, as described in claim 14, wherein said at least oxygen therapy treatment comprises air enriched with oxygen provided to said patient at a rate of 2-6 liters per minute.

17. The system for treating addictions, as described in claim 7, wherein said patient performs said dynamic physical exercises in morning sessions during an initial period after completion of said system's implementation.

18. The system for treating addictions, as described in claim 17, wherein said dynamic exercises are preceded by consumption of said particular dietary supplements.

19. The system for treating addictions, as described in claim 8, wherein said patient performs said static physical exercises in evening sessions during an initial period after completion of said system's implementation.

20. The system for treating addictions, as described in claim 19, wherein said static exercises are preceded by consumption of said particular dietary supplements.

21. The system for treating addictions, as described in claim 7, wherein patients with polymorphism in a DBH dopamine beta-hydroxylase gene perform only said dynamic exercises after an initial period following completion of said system's implementation.

22. The system for treating addictions, as described in claim 8, wherein patients with polymorphism in any of DRD2 dopamine D2 receptor, DAT dopamine transporter, or SERT serotonin transporter genes perform only said static exercises after an initial period following completion of said system's implementation.

23. The system for treating addictions, as described in claim 21, wherein said dynamic exercises are performed in a morning session and a short version of static exercises are performed in an evening session.

24. The system for treating addictions, as described in claim 22, wherein said static exercises are performed in an evening session and a short version of dynamic exercises are performed in a morning session.

25. The system for treating addictions, as described in claim 12, wherein said particular dietary supplements, which increase the production of neuromediators, are taken after an initial period following completion of said system's implementation.

26. The system for treating addictions, as described in claim 13, wherein said particular dietary supplements, which increase the production of the neurotransmitters, are taken after an initial period following completion of said system's implementation.
